(19) United States
(12) Patent Application Publication
(10) Pub. No.: US 2008/0051813 A1
(43) Pub. Date: Feb. 28, 2008

(54) Adapter Sleeve

(75) Inventor: Raymond Dunn, Shrewsbury, MA (US)

Correspondence Address: WEINGARTEN, SCHURGIN, GAGNEBIN & LEBOVICI LLP, TEN POST OFFICE SQUARE, BOSTON, MA, 02109, US

(73) Assignee: UNIVERSITY OF MASSACHUSETTS, Boston, MA (US)

(21) Appl. No.: 11/662,716

(22) PCT Filed: Sep. 13, 2005
(86) PCT No.: PCT/US05/32416

§ 371 Date: Sep. 26, 2007

Related U.S. Application Data
(60) Provisional application No. 60/609,430, filed on Sep. 13, 2004.

(51) Int. Cl. A61B 17/3211

(52) U.S. Cl. 606/167

(57) ABSTRACT

The present invention relates to an adapter sleeve that provides a contoured handle surface that is used to hold a scalpel blade during a surgical procedure. The adapter sleeve in a preferred embodiment can include a cavity in which the scalpel blade can be removably inserted for a single procedure. Alternatively, the adapter can be manufactured as a sterilizable unit with the scalpel blade capable of being rigidly mounted to a distal end of a handle.

### CROSS REFERENCE TO RELATED APPLICATIONS

This application claims the benefit under 35 U.S.C. 119(e) of U.S. Provisional Application No. 60/609,430, filed Sep. 13, 2004, the disclosure of which is incorporated by reference herein.

### STATEMENT REGARDING FEDERALLY SPONSORED RESEARCH OR DEVELOPMENT

N/A

### BACKGROUND

Surgeons have traditionally used scalpels for a variety of surgical procedures to cut or excise tissue. Surgical precision requires, in certain types of procedures, the angle of orientation of the scalpel blade to be of particular importance.

For example, each year many people have skin growths, such as moles, removed. In order to excise the mole the surgeon must use a scalpel to make two mirror image incisions in the skin around the mole. Depending on the “handedness” of the surgeon one incision is more difficult to make than the other. This is due to the traditional flat handle design used for most scalpels, which makes it much harder to hold the blade in the proper fashion. The flat handle requires the surgeon's wrist to roll more for one incision versus the other, thus increasing the chance of a minor hand tremor or misalignment of the blade relative to the surgical site while cutting. This in turn, reduces the precision with which mirror image symmetry can be created.

Thus, there is an ongoing need for improvements in scalpel design to facilitate greater ease in surgical applications.

### SUMMARY OF THE INVENTION

This present invention relates to an adapter sleeve that can be attached to a scalpel blade. The adapter sleeve provides a handle that is easier to hold, thereby giving the surgeon greater control and ability to make more precise incisions. The adapter sleeve of the present invention includes a cavity extending through a portion of the sleeve that receives the proximal end or handle of the scalpel such that the blade extends from the distal end of the adapter. An outer surface region of the adapter sleeve includes a handle portion that is gripped manually by the hand of the user during a surgical procedure. The sleeve handle has a contoured surface to provide for proper orientation of the blade of the scalpel relative to the surgical site. The contoured surface of the handle can include a proximal section with a thicker diameter that tapers to a smaller diameter section at the distal end. The distal section can have a recess for the thumb of the user. The blade can be rigidly oriented relative to the surface features to improve manual manipulation. The sleeve handle can be left or right handed.

In accordance with a preferred embodiment, the method for using the adapter sleeve of the present invention involves selecting a blade, inserting it into the adapter, performing the selected procedure, removing the scalpel for cleaning, sterilization, and reuse, and either sterilizing or disposing of the adapter sleeve.

In another preferred embodiment, the scalpel blade is assembled with the adapter sleeve during manufacture. The adapter sleeve in this embodiment is made of a plastic material suitable for sterilization after use. The sleeve can be molded as a unitary body with selected portions of the surface having a hatched, abraided or dimpled surface to provide frictional surface regions unlikely to slip when grasped by the user. The sleeve can also be molded in two pieces that snap together about the blade using a connector.

A preferred embodiment can also include a rotational mechanism to provide for adjustment of the angular orientation of the blade relative to the adapter handle. In one example, the cavity is sized to provide for two different angular orientation of the blade edge relative to the adapter handle, thus allowing the user to select the proper orientation for a given procedure. In a second example, the adapter handle can have inner and outer sections or sleeves that rotate relative to each other. The user can turn the blade relative to the handle which “clicks” between selectable rotational positions to find that most suitable.

The foregoing and other features and advantages of the system and method for a scalpel adapter sleeve will be apparent from the following more particular description of preferred embodiments of the system and method as illustrated in the accompanying drawings in which like reference characters refer to the same parts throughout the different views.

### BRIEF DESCRIPTION OF THE DRAWINGS

FIG. 1 is a front side perspective view of the adapter sleeve with a scalpel blade inserted therein in accordance with a preferred embodiment of the present invention.

FIG. 2 is a perspective view of the proximal end of the device seen in FIG. 1 in accordance with a preferred embodiment of the present invention.

FIG. 3 is a perspective view of the adapter sleeve with a scalpel blade positioned for insertion at the proximal end of the adapter in accordance with a preferred embodiment of the present invention.

FIG. 4 is a perspective view of the proximal end of the adapter and scalpel in accordance with a preferred embodiment of the present invention.

FIG. 5 is a rear side view of the adapter and scalpel in accordance with a preferred embodiment of the present invention.

FIG. 6 is a lateral side view of the adapter sleeve and scalpel in accordance with a preferred embodiment of the present invention.

FIG. 7 is a cross-section view illustrating an internal cavity that receives a scalpel blade in accordance with a preferred embodiment of the present invention.

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

### DETAILED DESCRIPTION OF THE INVENTION

This invention describes an adapter sleeve that can be attached to existing flat handled scalpels. The adapter sleeve can be elliptical in shape at least along a portion thereof and crosshatched or beveled to make the handle a more natural feel thereby making it easier to hold to provide superior scalpel manipulations. In addition, the design can also be manufactured as a knife handle unit.

The adapter sleeve can be a sterilizable or disposable, elliptical-shaped, beveled plastic handle that can be used on existing scalpel handles. In addition to making the handle of the scalpel easier to hold, the design of the adapter also has the advantage of making the handle less slippery than the traditional steel handle. Through use of the adapter sleeve the scalpel will be easier to manipulate thereby allowing the precision of the incision to be increased.

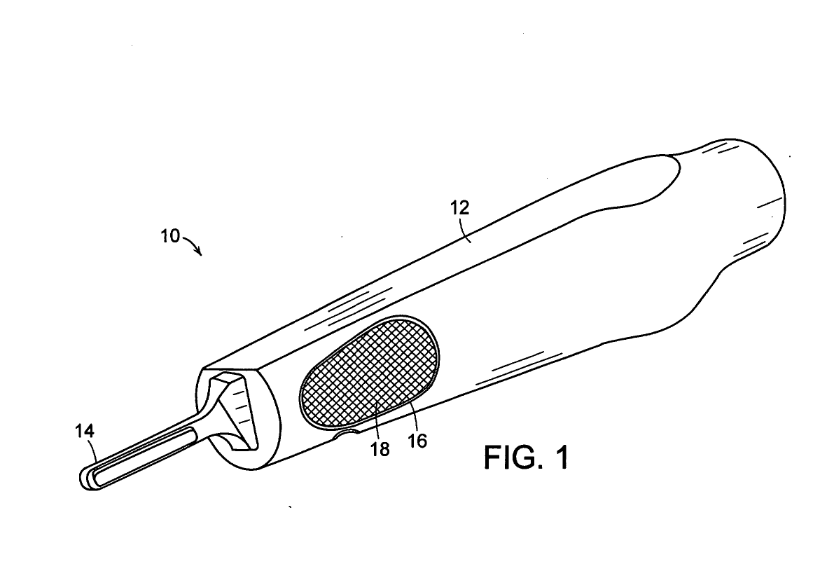

A preferred embodiment of the invention is illustrated in connection with FIG. 1. The adapter sleeve scalpel device 10 of this embodiment has a contoured handle 12 and a distally mounted blade 14 that is rigidly attached to the handle. A front side of the handle 12 is shown with a recess 16 sized to receive the thumb of a user. The recess can be a hatched, abraded, or provided with some other frictionally enhanced surface region 18 to improve gripping and manual manipulation of the blade.

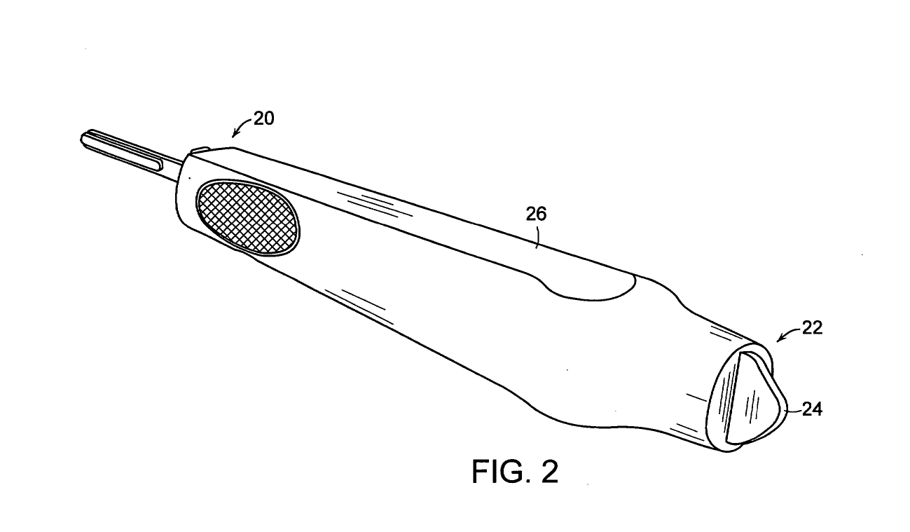

A rear perspective view of an adapter sleeve and scalpel is shown in FIG. 2. The adapter handle has a distal portion 20, a proximal portion 22, and a lateral side 26 which has a flattened portion in this embodiment. A proximal end 24 of the scalpel blade can extend from proximal portion 22.

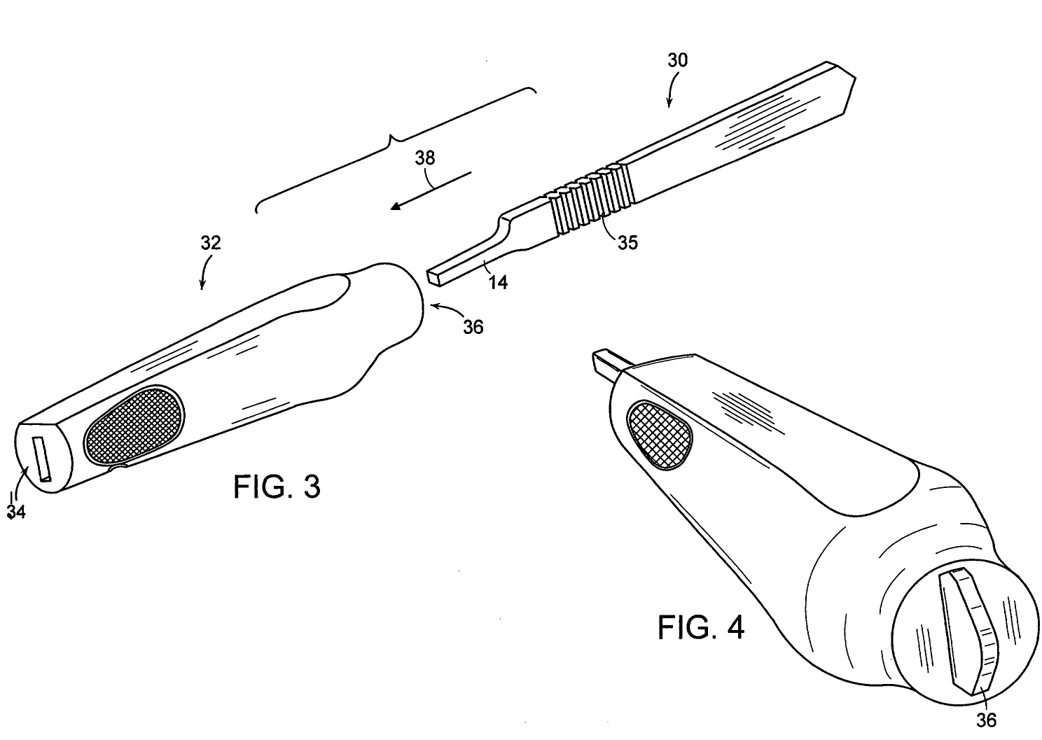

In the embodiment illustrated in FIG. 3, the distal end 14 of scalpel 30 is inserted in direction 38 into a proximal cavity opening 36 of the adapter sleeve 32. The scalpel blade slides through the cavity until the blade extends through the distal cavity or channel opening 34 of the adapter. The inner cavity surface is shaped to conform to the shape of the scalpel blade to securely grip the blade during use. The inner surface of the cavity can also be scalloped or dimpled to engage the frictional surface features 35 of the scalpel handle. Alternatively, the adapter handle can be fitted with a “snap” feature that locks the scalpel handle in place upon insertion. FIG. 4 shows a rear perspective view of the embodiment in FIG. 3 with the scalpel inserted into proximal opening 36.

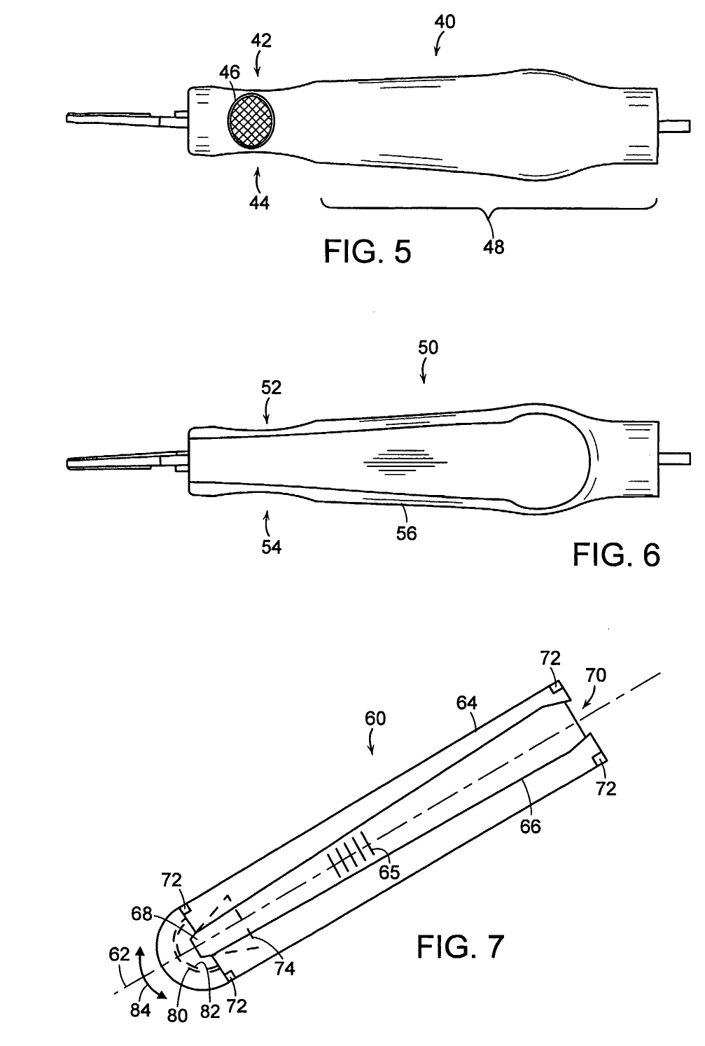

A preferred embodiment of the invention illustrated in FIG. 5 shows a side view in which the proximal portion 48 of adapter 40 has a circular or elliptical cross-sectional shape. The front and rear sides of adapter 40 have recesses 42 and 44, respectively, that can be gripped by the thumb and fingers of the user. Additionally, one or both lateral sides can have smaller recesses 46.

In the embodiment illustrated in FIG. 6, the adapter has front and rear recesses 52 and 54, respectively, however, the lateral sides have flattened regions 56. The features illustrated in FIGS. 5 and 6 give the user a positive feel for how the blade of the scalpel is oriented relative to the surgical sites.

A cross-sectional view of an adapter handle 60 is shown in FIG. 7. In a preferred embodiment, the adapter 60 is made of a solid unitary element 64, in which a cavity 66 extends along a longitudinal axis 62. The cavity 66 can be shaped to conform to a handle of a scalpel blade such as that shown at 30 in FIG. 3. The user, as described previously herein, inserts the distal end 14 of the blade through the proximal opening 70 until the blade 14 extends through the distal opening 68. The interior surface of the cavity can have a fractional surface such as ridges 65 that engage surface features 35 (FIG. 3) of the blade handle. In an embodiment in which the adapter handle has two sections, connector 72 can be used to connect the two portions together.

In another preferred embodiment, the scalpel blade has a blade section and a proximal section that mates with the internal cavity 74 shown in outline in FIG. 7. In this embodiment, the cavity does not extend through the length of the handle 64. Rather, The proximal section of the blade fits snugly within distal cavity 74 and the proximal section of the handle can be a solid material.

A preferred embodiment of the invention can employ the option of providing different angular orientations of the blade relative to the adapter handle. The different positions can be over a continuous range of angular position 84 between 0-180 degrees, or they can be discrete. In one embodiment, the cavity can be sized in a manner allowing for two discrete angular positions in which the blade can be inserted. In another embodiment, the adapter 60 can include an outer sleeve adapter section that fits at cylindrical surface 80 relative to an inner sleeve or section that nests at cylindrical surface 82 with the outer section. The two sections rotate 84 relative to each other such that the user can adjust between angular orientations of the blade relative to the adapter. The user can select between discrete angular orientations in which the user can “click” between fixed angular positions that are, for example, 10-30 degrees apart.

The claims should not be read as limited to the described order or elements unless stated to that effect. Therefore, all embodiments that come within the scope and spirit of the following claims and equivalents thereto are claimed as the invention.

1. An adapter for performing a surgical procedure with a blade comprising:
an adapter including a contoured handle surface to be held in a hand of a user; and
a cavity within the adapter to receive a surgical blade device such that a blade extends through a distal opening of the cavity.

2. The adapter of claim 1 wherein the adapter further comprises a proximal opening in the cavity such that a distal end of the blade can be inserted through the proximal opening.

3. The adapter of claim 1 wherein the contoured handle surface comprises a proximal portion having an elliptical shape.

4. The adapter of claim 1 wherein the contoured handle surface comprises a distal portion having a front side with a recess for a thumb of a user.

5. The adapter of claim 4 wherein the contoured handle surface further comprises a rear side having a second recess for a finger of a user.

6. The adapter of claim 1 wherein the contoured handle surface has a first lateral side and a second lateral side.

7. The adapter of claim 1 wherein the cavity conforms to a shape of a handle of the surgical blade.

8. The adapter of claim 1 wherein the handle surface has a frictional portion to be engaged by a finger of the user.

9. The adapter of claim 1 wherein the blade device comprises a blade having a proximal end affixed to a cavity at the distal end of the adapter.

10. The adapter of claim 1 wherein the adapter comprises a molded plastic material.

11. The adapter of claim 1 wherein the adapter comprises a first section that attaches to a second section with a connector.

12. A method of using a surgical blade comprising:
providing a surgical blade attached to an adapter, the adapter having a contoured handle surface;
manually grasping the contoured handle surface; and performing a surgical procedure with the surgical blade.

13. The method of claim 12 further comprising the step of inserting a blade handle into a cavity of the adapter.

14. The method of claim 12 further comprising rotating an inner adapter section relative to an outer adapter section to alter an orientation of the blade relative to the adapter surface.

15. The method of claim 12 further comprising providing a surgical blade having a body and a cutting surface at a distal tip of the body and inserting the distal tip into a proximal opening of the adapter.

16. The method of claim 15 further comprising positioning the cutting surface of the blade through a distal opening of the adapter.

17. The method of claim 16 wherein the cavity has a shape conforming to a blade handle.

18. The method of claim 12 further comprising providing a handle surface with a finger recess.

19. The method of claim 12 further comprising altering an angular orientation of the blade to the handle surface.

20. The method of claim 12 further comprising inserting a proximal end of the blade into a distal opening of the adapter.

21. An adapter for performing a surgical procedure with a blade comprising:
an adapter including a handle surface to be held in a hand of a user;
a cavity within the adapter to receive a surgical blade device, the cavity having a distal opening and a surgical blade having a body and a distal blade, the body being positioned within the cavity.

22. The adapter of claim 21 wherein the adapter further comprises a proximal opening in the cavity such that a distal end of the blade can be inserted through the proximal opening.

23. The adapter of claim 21 wherein the handle surface comprises a contoured proximal portion having an elliptical shape.

24. The adapter of claim 21 wherein the handle surface comprises a distal portion having a front side with a recess for a thumb of a user.

25. The adapter of claim 24 wherein the handle surface further comprises a rear side having a second recess for a finger of a user.

26. The adapter of claim 21 wherein the handle surface has a first lateral side and a second lateral side.

27. The adapter of claim 21 wherein the cavity conforms to a shape of a handle of the surgical blade.

28. The adapter of claim 21 wherein the handle surface has a frictional portion to be engaged by a finger of the user.

29. The adapter of claim 21 wherein the blade device comprises a blade having a proximal end affixed to a cavity at the distal end of the adapter.

30. The adapter of claim 21 wherein the adapter comprises a molded plastic material.

31. The adapter of claim 21 wherein the adapter comprises a first section that attaches to a second section with a connector.

32. A method of using a surgical blade comprising:
providing a surgical blade;
inserting the blade into an adapter having a handle surface;
manually grasping the handle surface; and
performing a surgical procedure with the surgical blade.

33. The method of claim 32 further comprising the step of inserting a blade handle into a cavity of the adapter.

34. The method of claim 32 further comprising rotating an inner adapter section relative to an outer adapter section to alter an orientation of the blade relative to the adapter surface.

35. The method of claim 32 further comprising providing an adapter having a molded unitary body.

36. The method of claim 32 further comprising providing an adapter having a first element that connects to a second element about the blade with a connector.

37. The method of claim 32 further comprising disposing of the adapter after a single use.

38. The method of claim 32 further comprising providing an adapter with a cavity extending through an entire length of the adapter.

39. The method of claim 32 further comprising providing an adapter that has an opening at a first end and is closed at a second end.

40. The method of claim 32 further comprising selecting an angle of orientation of the blade relative to the adapter at one of a continuous or discrete set of positions between 0 and 180 degrees.
